(19) United States
(12) Patent Application Publication
(10) Pub. No.: US 2009/0088373 A1
(43) Pub. Date: Apr. 2, 2009

(54) USE OF COMPOSITIONS TO ENHANCE INNATE IMMUNE RESPONSE

(76) Inventors: Richard L. Gallo, San Diego, CA (US); Thomas Hultsch, Mountain Lakes, NJ (US); Anton Stuetz, Vienna (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/236,074

(22) Filed: Sep. 23, 2008

Related U.S. Application Data
(60) Provisional application No. 60/975,992, filed on Sep. 28, 2007.

(51) Int. Cl. A61K 38/13; A61K 31/4353; A61K 31/215; A61K 31/519; A61K 31/407; A61K 31/593; A61K 31/4439; A61P 37/04

(52) U.S. Cl. 514/11; 514/291; 514/531; 514/532; 514/293; 514/411; 514/410; 514/167; 514/341

(57) ABSTRACT

The present invention discloses methods of using of compounds for the enhancement of the innate immune system of a patient. In particular, the active compounds of the present invention include at least one calcineurin inhibitor, mTOR inhibitor or non-immunosuppresive derivative, or a derivative, isomer, or pharmaceutically acceptable salt thereof; and optionally, at least one calciferol or LMW inhibitor, or a derivative, isomer, or pharmaceutically acceptable salt thereof. Pharmaceutical formulations comprising same are also disclosed.

### DESCRIPTION OF THE FIELD

Host protection against diseases caused by antigens are provided by the immune system, a collection of molecular and cellular mechanisms and processes which function synergistically to either rid the host of the offending agents or control their proliferation. The innate defense mechanisms of the immune system get involved early post-infection, and their function is to control the extent of infection in an agent nonspecific manner. The generation of acquired or adaptive immunity, with its antigen specificity and high degree of efficiency takes a period of time to develop. Individuals can vary greatly in the efficiency of their innate immune defenses. These differences can spell the difference between an infection which resolves prior to development of overt disease, and an infection which progresses to full-blown disease prior to the mobilization of the acquired immune defense mechanism.

The most important function of the immune system is to provide the ability to differentiate self from non-self and to protect the host from invasion by antigens. To carry out these tasks, a large and diverse array of effector mechanisms have evolved, the majority of which exhibit antigen specificity. Each individual effector mechanism possesses a degree of uniqueness with respect to its ability to influence the rate of progression, to detoxify, or to promote the elimination of microbial pathogens. Such diversity in available mechanisms is absolutely essential since no single effector response can effectively deal with all forms of pathogenic insults.

The different mechanisms employed to protect a host against antigens include physical barriers, phagocytic cells in the blood and tissues, natural killer cells and various blood-borne molecules. These mechanisms can repel, destroy or hold in check many types of antigens. Some of these defense mechanisms (a) are present prior to exposure to an antigen, (b) do not discriminate among most antigens, and (c) frequently cannot be sufficiently enhanced by such exposure. These defense mechanisms are the components of the innate immunity. Other defense mechanisms are induced or stimulated by exposure to antigens, are specific for distinct antigens and increase in magnitude and defensive capabilities with each successive exposure to a particular antigen. These mechanisms constitute adaptive (acquired) immunity.

Cutaneous innate immunity depends on two major components: 1) Pattern recognition receptors such as toll-like receptors (TLRs) and 2) effector molecules such as antimicrobial peptides (AMPs). In contrast to the adaptive immune system, innate immunity does not require specific pre-sensitization. The innate defense mechanisms of the immune system get involved early post-infection, and their function is to control the extent of infection in an agent nonspecific manner. Individuals can vary greatly in the efficiency of their innate immune defenses. In addition, the frequency and spectrum of multi-resistant species of pathogenic bacteria has dramatically increased over the past decade. Consequently, new strategies need to be pursued to meet the upcoming challenges that are being caused by the emergence of multi-drug resistance in bacteria and to assist individuals having an innate immune response which is not operating optimally.

Surface epithelia and resident microorganisms form the first barrier against pathogen invasion. The innate immune system provide the host with a constitutive or immediately inducible defense system that is capable of effectively dealing with the continuous attacks of a variety of pathogens at the mucosal epithelial surfaces. When antigens contact the epidermis, a cellular/biochemical cascade involving cells of the innate immune system is provoked. Comprised of a variety of effector cells and a suite of signal molecules, including lysozyme, lactoferrin, and the defensins, the innate immune system mobilizes a rapid response resulting in the inactivation and elimination of antigens, before adaptive immune mechanisms are mobilized in vivo. As such, molecules which upregulate the innate immune system where the presence of antigens are known or suspected represent ideal therapeutic candidates to replace antibiotics as the primary treatment modalities for various bacterial and viral based skin infections and diseases.

The incidence of antibiotic resistance is increasing rapidly to the point where some microbes are resistant to all of the present antibiotics known. This requires a careful choice of treatment as well as reducing the speed of treatment, because it may require testing to identify which antibiotic will be effective for treating the specific microbe. In addition, wide antibiotic use is further contributing to the problem of resistance. Thus, the need to identify new antibiotics is causing the price of these substances to be so high as to be prohibitive in some cases. Accordingly, there is a need for non-antibiotic treatment of infections in view of the problems associated with decades of antibiotic use and misuse.

### SUMMARY OF THE INVENTION

The present invention addresses these unmet needs by providing compositions, methods and uses which enhance the innate immune system response to treat infections and diseases. This treatment offers a number of advantages. Unlike existing antibiotics, these molecules are unlikely to induce antibiotic resistance. A further advantage is that these molecules, because they are produced by the host, will not induce allergic reactions. The method of the present invention is also more cost effective then that of antibiotic treatment.

This application relates to pharmaceutical compositions, methods and uses for treatment of diseases in an patient in which enhancement of the innate immune response can treat the disease, comprising administering to the patient a therapeutically effective amount of a composition comprising:

(a) at least one calcineurin inhibitor, mTOR inhibitor or non-immunosuppressive derivative, or a derivative, isomer, or pharmaceutically acceptable salt thereof; and

(b) optionally, at least one calciferol or LMW inhibitor, or a derivative, isomer, or pharmaceutically acceptable salt thereof.

### DETAILED DESCRIPTION OF THE INVENTION

As used herein, the term “active compound” refers to compounds which have an innate immune system enhancing effect. In particular, such compounds include calcineurin inhibitors, mTOR inhibitors, non-immunosuppresive derivatives, calciferols and LMW inhibitors.

As used herein, the term “derivative” refers to a chemically or biochemically modified active compound, resulting in the addition or substitution of a chemical moiety to such compound.

As used herein, the term “isomer” refers to active compound having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe active compounds are meant to be encompassed all possible stereoisomers.

As used herein, the term “optional” or “optionally” means that the subsequently described event or circumstance may or may not occur, and that the description includes instances where the event or circumstance occurs and instances in which it does not.

As used herein, the term “patient” refers to an animal, including humans.

As used herein, the term “pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

As used herein, the term “pharmaceutically acceptable salt” refers salts of active compounds which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

As used herein, the term “therapeutically effective amount” refers to that amount which, when administered to a patient for treating a disease, is sufficient to increase the innate immune system response in the patient

As used herein, the term “treatment” or “treating” means any administration of an active compound of the present invention and includes:

(1) preventing the disease from occurring in an patient which may be predisposed to the disease but does not yet experience or display the pathology or symptomatology of the disease,

(2) inhibiting the disease in an patient that is experiencing or displaying the pathology or symptomatology of the diseased (i.e., arresting further development of the pathology and/or symptomatology), or

(3) ameliorating the disease in a patient that is experiencing or displaying the pathology or symptomatology of the diseased (i.e., reversing the pathology and/or symptomatology).

The present invention includes methods for the treatment of diseases in an patient in which enhancement of the innate immune response can treat the disease, which method comprises administering to the patient a therapeutically effective amount of a composition comprising:
(a) at least one calcineurin inhibitor, mTOR inhibitor, or non-immunosuppresive derivative, or a derivative, isomer, or pharmaceutically acceptable salt thereof; and
(b) optionally, at least one calciferol or LMW inhibitor, or a derivative, isomer, or pharmaceutically acceptable salt thereof.

The present invention also includes pharmaceutical compositions for enhancing an innate immune response in a patient comprising a therapeutically effective amount of:
(a) at least one calcineurin inhibitor, mTOR inhibitor, or non-immunosuppressive derivative, or a derivative, isomer, or pharmaceutically acceptable salt thereof; and
(b) optionally, at least one calciferol or LMW inhibitor, or a derivative, isomer, or pharmaceutically acceptable salt thereof.

The present invention further includes methods for enhancing an innate immune response in a patient comprising administering to a patient a therapeutically effective amount of a composition comprising:
(a) at least one calcineurin inhibitor, mTOR inhibitor or non-immunosuppresive derivative, or a derivative, isomer, or pharmaceutically acceptable salt thereof; and
(b) optionally, at least one calciferol or LMW inhibitor, or a derivative, isomer, or pharmaceutically acceptable salt thereof.

Also disclosed herein is the use of a therapeutically effective amount of:
(a) at least one calcineurin inhibitor, mTOR inhibitor or non-immunosuppresive derivative, or a derivative, isomer, or pharmaceutically acceptable salt thereof; and
(b) optionally, at least one calciferol or LMW inhibitor, or a derivative, isomer, or pharmaceutically acceptable salt thereof;
   for the manufacture of a medicament for the stimulation of the innate immune response of a patient.

In another aspect of the invention, the calcineurin inhibitor may comprise tacrolimus, pimecrolimus, ascomycin, cyclosporin A, tacrolimus, cypermethrin, deltamethrin, fenvalerate, vocyclosporin and/or the derivatives, isomers and pharmaceutically acceptable salts thereof. In some aspects of the invention, the calcineurin inhibitors may comprise tacrolimus, pimecrolimus, ascomycin, and/or cyclosporin A.

In another aspect of the invention, the mTOR inhibitor may comprise rapamycin, rapamycin derivatives, everolimus, 7-tert-Butyl-6-(4-chloro-phenyl)-2-thioxo-2,3-dihydro-1H-pyrido[2,3-d]pyrimidin-4-one (SAB953), SAR943, 40-O-alkyl-rapamycin derivatives, 32-deoxo-rapamycin derivatives, 32-hydroxy-rapamycin derivatives, 32-deoxorapamycin, 16-O-substituted rapamycin derivatives, 16-pent-2-ynyloxy-32-deoxorapamycin, 16-pent-2-ynyloxy-32(S or R) -dihydro-rapamycin, 16-pent-2-ynyloxy-32(S or R)-dihydro-40-O-(2-hydroxyethyl)-rapamycin, rapamycin derivatives which are acylated at the oxygen group in position 40,40-[3-hydroxy-2-(hydroxy-methyl)-2-methylpropanoate]-rapamycin, rapamycin derivatives which are substituted in 40 position by heterocyclyl, 40-epi-(tetrazolyl)-rapamycin, rapalogs, 40-O-dimethylphosphinyl-rapamycin, AP23573, biolimus, 40-O-(2-ethoxy)ethyl-rapamycin (RAD001), TAFA-93, AP23464, AP23675, AP23841, and the derivatives, isomers and pharmaceutically acceptable salts thereof. In some aspects of the invention, the mTOR inhibitors may comprise SAB953, SAR943, rapamycin and RAD001.

In another aspect of the invention, the non-immunosuppresive derivative may comprise compounds of formula I to III

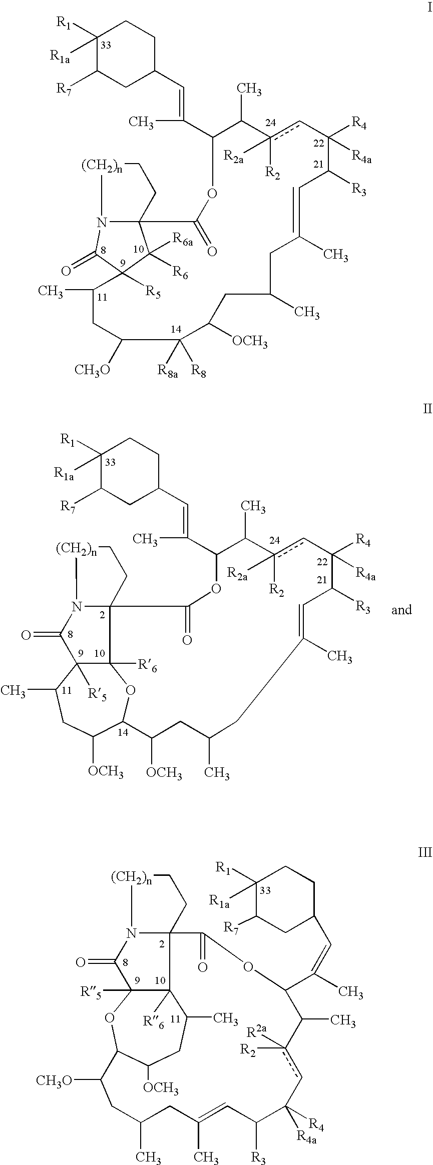

wherein the symbol [symbol] represents a single bond or, when R₂ₐ is absent, a double bond;

R₁ represents an optionally protected hydroxy group and R₁ₐ represents hydrogen;

or R₁ and R₁ₐ together represent oxo;

R₂ represents an optionally protected hydroxy group or together with R₄forms the —OC(═O)O— group, and R₂ₐ represents hydrogen or is absent;

whereby when the symbol [symbol] is a single bond,

R₂ together with R₂ₐ also represents oxo;

R₃ represents methyl, ethyl, n-propyl or allyl;

R₄ represents optionally protected hydroxy or together with R₂ forms the —OC(═O)O— group, and

R₄ₐ represents hydrogen;

or R₄ together with R₄ₐ represents oxo;

R₅ represents alkoxycarbonyloxy, halogen, optionally protected hydroxy, lower alkoxy, acyloxy or a group —OC(═X)N(R₁₀)R₁₁;

or R₅together with R₆ₐ forms a group —OC(═X)N(R′₁₀)— attached with the nitrogen atom to the carbon atom carrying R₆ₐ, whereby X represents oxygen or sulfur, R₁₀ and R₁₁ independently represent hydrogen or lower alkyl or together with the nitrogen atom form a five- or six-membered ring optionally containing a second heteroatom such as nitrogen or oxygen, and R′₁₀ is hydrogen or lower alkyl;

or R₅ together with R₈ₐ represents oxy, whereby R₈ represents hydroxy;

R₆ represents hydroxy, and R₆ₐ represents hydrogen or together with R₅ forms a group —OC(═X)N(R′₁₀)— as defined above;

or R₆and R₆ₐ together represent oxo;

R₆ represents optionally protected hydroxy, lower alkoxy or acyloxy and R′₆represents hydroxy;

or R′₅ and R′₆ together form the —OC(═O)O— group;

R″₅ represents hydroxy or lower alkoxy and R″.sub.6 represents hydroxy;

or R″₅and R″₆ together form the —OC(═O)O— group;

R₇represents methoxy or hydroxy;

R₈ represents an optionally protected hydroxy group, acyloxy, imidazolylcarbonyloxy or alkoxycarbonyloxy and R₈ₐ represents hydrogen;

or R₈ represents hydroxy and R₈ₐ together with R₅ represents oxy;

or R₈ together with R₈ₐ represents oxo; and

n represents 1 or 2;

and the derivatives, isomers and pharmaceutically acceptable salts thereof.

In another aspect of the invention, the calciferol may comprise calciferol, calcipotriol, calcitriol, cholecalciferol, 22,23-dihydroergocalciferol, 25-hydroxycholecalciferol, 25-hydroxyergocalciferol, maxacalcitol, falecalcitol, falecalcitriol, tacalcitol, and the derivatives, isomers and pharmaceutically acceptable salts thereof.

In another aspect of the invention, the LMW inhibitor is a compound of the formula

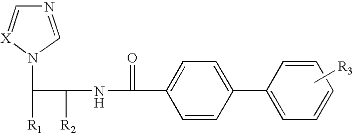

wherein either R₁ is phenyl, naphthyl, thienyl or pyridyl, or phenyl, naphthyl, thienyl or pyridyl monosubstituted by halogen, C₁-C₄ alkoxy, C₁-C₄ alkyl, di-C₁-C₄ alkylamino or cyano and R₂ is hydrogen; or

R₁ is hydrogen and

R₂ is pyridyl or 2-(5-chloro)pyridyl;

R₃ is hydrogen, halogen, C₁-C₄ alkyl, C₁-C₄ alkoxy, cyano, C₁-C₄ alkoxycarbonyl, C₁-C₄ alkylcarbonyl, amino or di-C₁-C₄ alkylamino; and

X is CH;

and the derivatives, isomers and pharmaceutically acceptable salts thereof.

Diseases which may be treated using the methods, uses and compositions of the present invention include cancers, acne, atopic dermatitis, wound infections, MRSA, viral skin infections, HSV, HPV, Poxvirus, onychomycosis, viral and bacterial infections of the oro-pharynx and respiratory tract, RSV, mycoplasm, infections of urinary tract, Chlamydia, viral and bacterial infections of the GI tract, psoriasis, contact dermatitis, eczematous dermatitises, seborrhoeic dermatitis, Lichen planus, Pemphigus, bullous Pemphigoid, Epidermolysis bullosa, urticaria, angioedemas, vasculitides, erythemas, cutaneous eosinophilias, Lupus erythematous, Alopecia areata, cornefied epithelia, gingivitis, oro-pharyngitis, inflammatory bowel disease, Crohns disease, recurrent vaginitis (E. coli), urethritis)), and conjunctiva.

It has surprisingly been found that calcineurin inhibitors, mTOR inhibitors (or “immunophylin ligands”) and non-immunosuppressive derivatives may be used for the enhancement of the innate immune system and thereby treatment of various skin infections and diseases, and further, that combinations of a calcineurin inhibitor and an mTOR inhibitor have a synergistic enhancing effect. It has also surprisingly been found that the use of an LMW inhibitor, calciferol or combinations thereof co-administered with at least one calcineurin inhibitor and mTOR inhibitor has a synergistic enhancement effect on the innate immune system. The administration of these compounds unexpectedly augments the anti-pathogenic activity of the innate immune system. This effect is expected to be of particular clinical benefit for patients with an impaired innate immune response, such as subjects with atopic dermatitis.

It has also surprisingly been found that low doses of the compositions of the present invention produce an enhancement of the innate immune system. Treatment of patients with these low concentrations of active compound produces an unexpected and unusual bell-shaped dose response curve. For example, treatment of patients with 10 and 20 nanomolar concentrations of pimecrolimus creme unexpectedly produced such a curve. The ability to use low doses of some macrolactams, such as the calcineurin inhibitor pimecrolimus, to treat certain diseases, such as atopic dermatitis, is also surprising as such compounds are known to be ineffective regarding anti-inflammatory effects where a low concentration is administered in a topical preparation. In contrast with standard treatment regimens, this enables much lower or less frequent dosing, which decreases potential toxicity of treatment and thus increases treatment safety.

Calcineurin plays a pivotal role in the T cell receptor (TCR)-mediated signal transduction pathway, leading to the transcriptional activation of certain cytokines, e.g., IL-2, which are required for an immune response. Calcineurin is a calcium and calmodulin-dependent protein serine/threonine phosphatase. Calcineurin has been shown to modulate the activity of several transcription factors that bind to the IL-2 promoter, including NF-AT, NF-κB and AP-1. It has been reported that calcineurin dephosphorylates the cytoplasmic subunit of NF-AT, allowing it to translocate into the nucleus to activate transcription.

Calcineurin inhibitors are known and described in, for example, U.S. Pat. Nos. 6,686,450; 6,492,325; 6,046,005; 5,807,693; 5,774,354; 5,723,436; and 5,629,163; and in U.S. Patent Application Nos. 20050008640; 20040224876; 20040091477; 20040033941; 20030045679; and 20020019344. Specific examples include, but are not limited to, cyclbsporin A, tacrolimus, ascomycin, pimecrolimus, cypermethrin (cyclopropanecarboxylic acid, 3-(2,2-dichloroethenyl)-2,2-dimethyl-,cyano(3-phenoxyphenyl)methyl ester), deltamethrin (cyclopropanecarboxylic acid, 3-(2,2-dibromoethenyl)-2,2-dimethyl-(S)-cyano(3-phenoxyphenyl)methyl ester, (1R,3R)), fenvalerate(benzeneacetic acid, 4-chloro-α-(1-methylethyl)-cyano(3-phenoxyphenyl)methyl ester) and vocyclosporin.

The calcineurin inhibitor may be administered alone, combined with at least one additional calcineurin inhibitor, mTOR inhibitor or non-immunosuppressive derivative, and/or combined with calciferols and/or LMW inhibitors. It has been unexpectedly discovered that a combination of at least one calcineurin inhibitor and/or LMW inhibitor and at least one calciferol or LMW inhibitor has a synergistic effect on the induction of the innate immune system. Accordingly, the calcineurin inhibitors and/or mTOR inhibitors and calciferol and/or LMW inhibitors are preferably administered to the patient in a synergistic amount (e.g., the combined treatment effect of the two active compounds together is greater than the sum of the effect of the two active compounds when administered individually) or the calciferol and/or LMW inhibitors administered in an amount effective to enhance the activity of the calcineurin inhibitor and/or LMW inhibitor in treating the disease or condition for which the active compounds are being administered.

mTOR inhibitors may be administered alone, in combination with other mTOR inhibitors, calcineurin inhibitors, or non-immunosuppresive derivatives and/or in combination with at least one calciferol or LMW inhibitor. m-TOR inhibitors include, but are not limited to, rapamycin, rapamycin derivatives, everolimus, 7-tert-Butyl-6-(4-chloro-phenyl)-2-thioxo-2,3-dihydro-1H-pyrido[2,3-d]pyrimidin-4-one, SAR943, 40-O-alkyl-rapamycin derivatives, 32-deoxo-rapamycin derivatives and 32-hydroxy-rapamycin derivatives, such as 32-deoxorapamycin, 16-O-substituted rapamycin derivatives such as 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, rapamycin derivatives which are acylated at the oxygen group in position 40, e.g. 40-[3-hydroxy-2-(hydroxy-methyl)-2-methylpropanoate]-rapamycin (CC1779), rapamycin derivatives which are substituted in 40 position by heterocyclyl, e.g. 40-epi-(tetrazolyl)-rapamycin (ABT578), the so-called rapalogs, e. g. as disclosed in WO9802441 or WO0114387, e.g. such as 40-O-dimethylphosphinyl-rapamycin, including AP23573, and compounds disclosed under the name biolimus (biolimus A9), including 40-O-(2-ethoxy)ethyl-rapamycin, and compounds disclosed under the name TAFA-93, AP23464, AP23675 or AP23841.

Non-immunosuppressive derivatives include, but are not limited to, compounds disclosed in U.S. Pat. No. 5,514,685 of the formula I to II

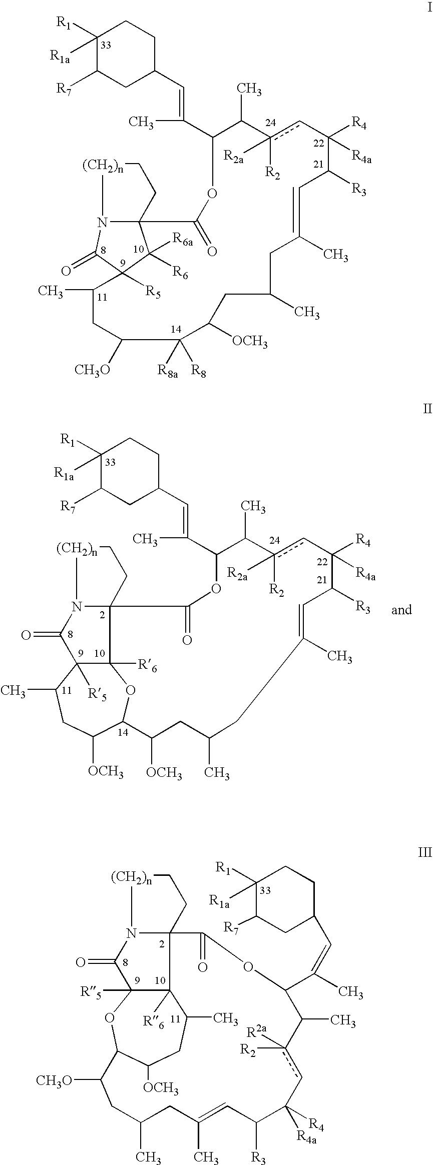

wherein the symbol [symbol] represents a single bond or, when R₂ₐ is absent, a double bond;

R₁ represents an optionally protected hydroxy group and R₁ₐ represents hydrogen;

or R₁ and R₁ₐ together represent oxo;

R₂ represents an optionally protected hydroxy group or together with R₄forms the —OC(═O)O— group, and R₂ₐ represents hydrogen or is absent;

whereby when the symbol [symbol] is a single bond,

R₂ together with R₂ₐ also represents oxo;

R₃ represents methyl, ethyl, n-propyl or allyl;

R₄ represents optionally protected hydroxy or together with R₂ forms the —OC(═O)O— group, and

R₄ₐ represents hydrogen;

or R₄ together with R₄ₐ represents oxo;

R₅ represents alkoxycarbonyloxy, halogen, optionally protected hydroxy, lower alkoxy, acyloxy or a group —OC(═X)N(R₁₀)R₁₁;

or R₅ together with R₆ₐ forms a group —OC(═X)N(R′₁₀)— attached with the nitrogen atom to the carbon atom carrying R₆ₐ, whereby X represents oxygen or sulfur, R₁₀ and R₁₁ independently represent hydrogen or lower alkyl or together with the nitrogen atom form a five- or six-membered ring optionally containing a second heteroatom such as nitrogen or oxygen, and R′₁₀ is hydrogen or lower alkyl;

or R₅ together with R₈ₐ represents oxy, whereby R₈ represents hydroxy;

R₆ represents hydroxy, and R₆ₐ represents hydrogen or together with R₅ forms a group —OC(═X)N(R′₁₀)— as defined above;

or R₆and R₆ₐ together represent oxo;

R′₅ represents optionally protected hydroxy, lower alkoxy or acyloxy and R′₆represents hydroxy;

or R′₅ and R′₆ together form the —OC(═O)O— group;

R″₅ represents hydroxy or lower alkoxy and R″.sub.6 represents hydroxy;

or R″₅and R″₆ together form the —OC(═O)O— group;

R₇represents methoxy or hydroxy;

R₈ represents an optionally protected hydroxy group, acyloxy, imidazolylcarbonyloxy or alkoxycarbonyloxy and R₈ₐ represents hydrogen;

or R₈ represents hydroxy and R₈ₐ together with R₅ represents oxy;

or R₈ together with R₈ₐ represents oxo; and

n represents 1 or 2;

and the derivatives, isomers and pharmaceutically acceptable salts thereof.

Non-immunosuppressive derivatives include 1E-(1R,3R,4R)]1R,4S,5R,6S,9R,10E,13S,15S,16R,17S,9S,20S-9-ethyl-6,16,20-trihydroxy-4-[2-(4-hydroxy-3-methoxycyclohexyl)-1-methylvinyl]-15,17-dimethoxy-5,11,13,19-tetramethyl-3-oxa-22-azatricyclo[18.6.1.0(1,22)]heptacos-10-ene-2,8,21,27-tetraone (ASD732).

Calciferol is a fat soluble vitamin which is essential as a positive regulator of calcium homeostasis, modulation of immune response, modulation of the process of insulin secretion by the pancreatic B cell, muscle cell function, and the differentiation and growth of epidermal and hematopoietic tissues, and has been shown to induce innate immune responses.

Calciferols include, but are not limited to, calciferol, the synthetic form of vitamin D (vitamin D2; ergocalciferol; Deltalin®), calcipotriol (Daivonex®; calcipotriene), calcitriol (1α,25-dihydroxycholecalciferol; 1α,25-dihydroxyvitamin D3; Rocaltrol®), cholecalciferol (vitamin D3; Trivitan®), 22,23-dihydroergocalciferol (vitamin D4; 22,23-dihydrovitamin D2), 25-hydroxycholecalciferol, 25-hydroxyergocalciferol, maxacalcitol, falecalcitol or falecalcitriol (ST-630; F6VD3; flocalcitriol; Penedrem®) and tacalcitol (1α,24R-dihydroxycholecalciferol; 1α,24R-dihydroxyvitamin D3; Bonalfa®).

LMW inhibitors within in the scope of the present invention are those LMW inhibitors which function to inhibit the metabolism of calciferol, thereby increasing the concentration in skin of calciferol and inducing the innate immune response.

LMW inhibitors include, but are not limited to, compounds as disclosed in U.S. Pat. No. 5,622,982, of the formula

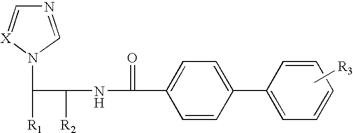

wherein either R₁ is phenyl, naphthyl, thienyl or pyridyl, or phenyl, naphthyl, thienyl or pyridyl monosubstituted by halogen, C₁-C₄ alkoxy, C₁-C₄ alkyl, di-C₁-C₄ alkylamino or cyano and

R₂ is hydrogen; or

R₁ is hydrogen and

R₂ is pyridyl or 2-(5-chloro)pyridyl;

R₃ is hydrogen, halogen, C₁-C₄alkyl, C₁-C₄ alkoxy, cyano, C₁-C₄ alkoxycarbonyl, C₁-C₄ alkylcarbonyl, amino or di-C₁-C₄ alkylamino; and

X is CH;

and the derivatives, isomers and pharmaceutically acceptable salts thereof.

LMW inhibitors specifically include N-[4-(4-chlorophenyl)benzoyl]-2-(1H-imidazol-1 -yl)-2-phenyl-1-aminoethyane and the 2(S)- and 2(R)-enantiomers of the same.

In another aspect of the invention, at least one of tacrolimus, pimecrolimus, ascomycin, ASD732, and/or cyclosporin A, are combined with at least one calcitrol, especially vitamin D and/or vitamin D3.

In another aspect of the invention, at least one of SAB953, SAR943, rapamycin and/or RAD001 are combined with at least one calcitrol, especially vitamin D and/or vitamin D3.

The active compounds described above are useful for enhancing the innate immune system response and for the treatment of various conditions, including diseases for which stimulation of an innate immune response against microbials is expected to prevent the clinical manifestation or the aggravation of the disease. Non-limiting examples of such diseases include but are not limited to: acne (propione bacterim acnes) atopic dermatitis (S. aureus), wound infections (such as MRSA), viral skin infections (HSV, HPV and Poxvirus), onychomycosis (T. rubrum, T. mentagrophytes) viral and bacterial infections of the oro-pharynx and respiratory tract (such as RSV and mycoplasm), infections of the urinary tract (such as Chlamydia) and infections of the GI tract via pathogenic bacteria or viruses (such as Rotavirus).

The active compounds are also useful for the treatment of diseases of the epithelium which are associated with a deficiency/impairment of the epithelium's innate immune system to respond to microbial antigens, such as fungi, viruses, bacteria, or cancer signal cells, especially in atopic patients. Non-limiting examples of such diseases include cancer, especially skin cancer, cornefied epithelia like skin and adnexes (such as atopic dermatitis, psoriasis, nail-matrix and plate), mucosal epithelial surfaces like oral cavity, esophagus gastrum and gut (such as gingivitis, oro-pharyngitis, inflammatory bowel disease, Crohns disease), respiratory tract (nasal, tracheal, and bronchial epithelia), uro-gential tract (recurrent vaginitis (e coli), urethritis)), and the eye (conjunctiva).

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, for example, a pharmaceutically acceptable carrier. The pharmaceutical formulations may, for example, consist of from about 0.0001% to 100%, preferably from about 0.01% to about 80% of the active compound by weight of the formulation. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. These are prepared in a manner which is known per se, for example using conventional mixing, granulation, coating, solubilizing or lyophilizing processes. In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavored base, such as sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline or water-for-injectioh immediately prior to use. Extemporaneous injection solutions and suspensions may be prepared from sterile powders, granules and tablets of the kind previously described. For example, in one aspect of the present invention, there is provided an injectable, stable, sterile composition comprising an active compound as described above, in a unit dosage form in a sealed container. The compound or salt is provided in the form of a lyophilizate which is capable of being reconstituted with a suitable pharmaceutically acceptable carrier to form a liquid composition suitable for injection thereof into a patient. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compounds described herein, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

As noted above, the present invention provides pharmaceutical formulations comprising the active-compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.0001 or 1 to about 50 or 100 mg/kg of each active compound may be used, with all weights being calculated based upon the weight of the active compound, including the cases where a salt is employed. A dosage from about 1.0 ug/kg to about 50 or 100 mg/kg of each active compound may be employed for oral administration. Typically, a dosage from about 0.5 ug/kg to 5 or 10 mg/kg of each active compound may be employed for intramuscular injection.

For these indications the appropriate dosage will, of course, vary depending upon, for example, the host, the mode of administration and the nature and severity of the condition being treated.

The present invention is explained in greater detail in the following non-limiting Examples.

### EXAMPLE 1

To assess the effect of pimecrolimus on the innate immune response of the epidermis against bacteria, cultures of normal human keratinocytes (NHEK) are stimulated with a concentration of 10 nM pimecrolimus, 1 nM 1.25D3 (active 1.25 D-hydroxy calciferol) or their combination. Cells are lysed via sonication 20 minutes in an ice bath, after which cell lysates were mixed with Staphylococcus aureus ΔmprF cultures and incubated at 37° C. Bacterial growth over time is monitored by OD600 (Table 1). To determine if Pimecrolimus alone was able to kill Staphylococcus aureus ΔmprF, Pimecrolimus in a concentration of 10 nM is co-incubated with Staphylococcus aureus ΔmprF cultures and incubated at 37° C. (Table 2). Bacterial growth is monitored by OD600 (*: p<0.05; **: p<0.01; Student's t-test). Results are detailed in the tables below.

TABLE 1

| | | | | S. aureus and | | |
|---|---|---|---|---|---|---|
| | S. aureus | | | Keratinocyte Lysates (S + K) | | |
| Time | Mean | SD | N | Mean | SD | N |
| 0 h | 0.258000 | 0.003000 | 3 | 0.225000 | 0.01410674 | 3 |
| 2 h | 0.2706667 | 0.00450925 | 3 | 0.2576667 | 0.002081666 | 3 |
| 4 h | 0.350000 | 0.02773085 | 3 | 0.3116667 | 0.02663331 | 3 |
| 5 h | 0.4826667 | 0.09914299 | 3 | 0.3783333 | 0.03066486 | 3 |
| 6 h | 0.6486667 | 0.01871719 | 3 | 0.5736667 | 0.02272297 | 3 |
| 8 h | 0.8303334 | 0.1257789 | 3 | 0.778000 | 0.1145949 | 3 |
| | S + K + Pimecrolimus | | | S + K + 1.25D3 | | |
| Time | Mean | SD | N | Mean | SD | N |
| 0 h | 0.2253333 | 0.006658328 | 3 | 0.2203333 | 0.002886751 | 3 |
| 2 h | 0.2556667 | 0.001527525 | 3 | 0.2676667 | 0.008326664 | 3 |
| 4 h | 0.2806667 | 0.004618802 | 3 | 0.301000 | 0.01345362 | 3 |
| 5 h | 0.343000 | 0.004582576 | 3 | 0.3963333 | 0.05600297 | 3 |
| 6 h | 0.4506667 | 0.01607275 | 3 | 0.513000 | 0.07114071 | 3 |
| 8 h | 0.6843333 | 0.09047283 | 3 | 0.620333 | 0.020257 | 3 |

| | S + K + Pimecrolimus + 1.25D3 | | |
|---|---|---|---|
| Time | Mean | SD | N |
| 0 h | 0.2523333 | 0.006350853 | 3 |
| 2 h | 0.275000 | 0.02343075 | 3 |
| 4 h | 0.289000 | 0.003464102 | 3 |
| 5 h | 0.3713333 | 0.02909181 | 3 |
| 6 h | 0.4613333 | 0.0829538 | 3 |
| 8 h | 0.557000 | 0.1280352 | 3 |

This demonstrates that Pimecrolimus increases the ability of keratinocytes to kill Staphylococcus aureus ΔmprF. Further, the combination of Pimecrolimus and 1.25 D3 has an even greater antimicrobial effect than Pimecrolimus alone.

TABLE 2

| | S. aureus | | | S. aureus + Pimecrolimus | | |
|---|---|---|---|---|---|---|
| Time | Mean | SD | N | Mean | SD | N |
| 0 h. | 0.349000 | 0.03143247 | 3 | 0.3083333 | 0.004725816 | 3 |
| 2 h. | 0.3726667 | 0.02318045 | 3 | 0.3386667 | 0.001527525 | 3 |
| 4 h. | 0.409000 | 0.03051229 | 3 | 0.4156667 | 0.01258306 | 3 |
| 5 h. | 0.422000 | 0.01873499 | 3 | 0.4333333 | 0.02218859 | 3 |
| 6 h. | 0.460000 | 0.020000 | 3 | 0.4226667 | 0.008962886 | 3 |
| 8 h. | 0.6376666 | 0.04411727 | 3 | 0.601000 | 0.08107404 | 3 |

This demonstrates that Pimecrolimus does not have an antimicrobial effect by itself.

### EXAMPLE 2

To assess the effect of pimecrolimus on the innate immune response of the epidermis, cultures of normal human keratinocytes (NHEK) are stimulated with combinations of a concentration of 10 nM pimecrolimus, 1 μg/ml TLR 2/CD14 ligand—peptidoglycan (PGN), 10 μg/ml lipoteichoic acid (LTA), 2 0.1 μg/ml TLR 2/6 ligand—Malp-, and/or 0.1 μg/ml TLR 4/CD14 ligand—lipopolysaccharide (LPS). Expression of cathelicidin mRNA (Table 3) and protein as well as Human Beta Defensin (HBD) 2 (Table 4) and HBD3 (Table 5) expression in normal human keratinocytes is measured. Results are detailed in the tables below.

TABLE 3
(Cathelicidin)

| | without | | | | | |
|---|---|---|---|---|---|---|
| | Pimecrolimus | | | with Pimecrolimus | | |
| Treatment | Mean | SD | N | Mean | SD | N |
| control | 0.95 | 0.10 | 3 | 2.64 | 0.33 | 3 |
| PGN | 1.18 | 0.05 | 3 | 9.81 | 0.69 | 3 |
| LTA | 3.82 | 1.48 | 3 | 18.56 | 0.61 | 3 |
| Malp-2 | 2.29 | 0.46 | 3 | 26.08 | 2.86 | 3 |
| LPS | 0.65 | 0.10 | 3 | 8.67 | 0.52 | 3 |

TABLE 4
(HBD2)

| | without Pimecrolimus | | | with Pimecrolimus | | |
|---|---|---|---|---|---|---|
| Treatment | Mean | SD | N | Mean | SD | N |
| control | 1.04 | 0.35 | 3 | 6.53 | 1.940 | 3 |
| PGN | 3.26 | 0.29 | 3 | 0.51 | 4.430 | 3 |
| LTA | 4.43 | 1.10 | 3 | 1.77 | 0.530 | 3 |
| Malp-2 | 119.16 | 28.75 | 3 | 867.92 | 114.580 | 3 |
| LPS | 5.68 | 0.61 | 3 | 1.34 | 0.710 | 3 |

TABLE 5
(HBD3)

| | without | | | | | |
|---|---|---|---|---|---|---|
| | Pimecrolimus | | | with Pimecrolimus | | |
| Treatment | Mean | SD | N | Mean | SD | N |
| control | 1.02 | 0.22 | 3 | 0.72 | 0.503 | 3 |
| PGN | 0.61 | 0.03 | 3 | 1.24 | 0.327 | 3 |
| LTA | 2.46 | 0.24 | 3 | 0.72 | 0.120 | 3 |
| Malp-2 | 3.51 | 0.55 | 3 | 28.59 | 5.286 | 3 |
| LPS | 1.39 | 0.21 | 3 | 1.06 | 0.022 | 3 |

This demonstrates that Pimecrolimus increases transcript and protein abundance of antimicrobial genes in the presence of Toll 2/6 ligand Malp-2 in normal human keratinocytes.

### EXAMPLE 3

To assess the effect of pimecrolimus on the innate immune response of the epidermis, cultures of normal human keratinocytes (NHEK) are stimulated with a combination of a concentration of 10 nM pimecrolimus and of 1 nM 1.25D3. Gene expression of TLR1, TLR2, TLR4, TLR6 and CD14 are evaluated by qPCR. For CD14 protein expression, a Western Blot is performed and bands are quantified by densitometry using lmagej. Vehicle treated keratinocytes serve as controls. Cathelicidin protein expression is evaluated by immunofluorescence staining. Results of mRNA expression are shown in tables below.

TABLE 6

| Gene | Mean | SD | N | Mean | SD | N |
|---|---|---|---|---|---|---|
| | control | | | 1.25D | | |
| TLR1 | 1.01 | 0.060 | 3 | 1.93 | 0.18 | 3 |
| TLR2 | 1.14 | 0.660 | 3 | 0.66 | 0.14 | 3 |
| TLR4 | n.d. | | | n.d | | |
| TLR6 | 1.24 | 0.440 | 3 | 1.01 | 0.68 | 3 |
| CD14 | 1.03 | 0.220 | 3 | 3.01 | 0.61 | 3 |
| | Pimecrolimus | | | 1.25D + Pimecrolimus | | |
| TLR1 | 3.16 | 1.11 | 3 | 2.37 | 0.52 | 3 |
| TLR2 | 1.80 | 0.01 | 3 | 3.94 | 1.21 | 3 |
| TLR4 | n.d. | | | n.d. | | |
| TLR6 | 4.40 | 1.63 | 3 | 2.57 | 1.08 | 3 |
| CD14 | 2.07 | 0.22 | 3 | 62.06 | 14.37 | 3 |

n.d. = not detectable

### EXAMPLE 4

To assess the effect of pimecrolimus on the innate immune response of the epidermis, cultures of normal human keratinocytes (NHEK) are stimulated with a concentration of 10 nM pimecrolimus,1 nM 1.25 D3, 0.1 μg/ml TLR2/6 ligand Malp-2 or their combination. Expression of cathelicidin, CD14 and TLR2 mRNA normal human keratinocytes is measured and evaluated accordingly to the procedures as detailed in Example 2 above. Results are detailed in the tables below.

TABLE 7
(cathelicidin)

| | Mean | SD | N |
|---|---|---|---|
| control | 1.00 | 0.05 | 3 |
| 1.25D3 | 2.76 | 0.29 | 3 |
| PIM | 22.57 | 15.41 | 3 |
| 1.25D3 + PIM | 38.84 | 16.73 | 3 |
| Malp-2 + 1.25D3 | 120.09 | 11.51 | 3 |
| Malp-2 + 1.25D3 + PIM | 2191.47 | 355.50 | 3 |

TABLE 8
(CD14)

| | Mean | SD | N |
|---|---|---|---|
| control | 1.03 | 0.22 | 3 |
| 1.25D3 | 3.01 | 0.61 | 3 |
| PIM | 2.07 | 0.22 | 3 |
| 1.25D3 + PIM | 98.77 | 64.39 | 3 |
| Malp-2 + 1.25D3 | 3.59 | 0.69 | 3 |
| Malp-2 + 1.25D3 + PIM | 259.58 | 59.58 | 3 |

TABLE 9
(TLR2)

| | Mean | SD | N |
|---|---|---|---|
| control | 1.02 | 0.27 | 3 |
| 1.25D3 | 0.10 | 0.02 | 3 |
| PIM | 0.29 | 0.09 | 3 |
| 1.25D3 + PIM | 1.18 | 0.37 | 3 |
| Malp-2 + 1.25D3 | 3.59 | 0.69 | 3 |
| Malp-2 + 1.25D3 + PIM | 5.05 | 0.12 | 3 |

Combined, this demonstrates that Pimecrolimus enhances cathelicidin transcript abundance in the presence of low dose 1,25D3 and Malp-2 while simultaneously increasing CD14 and TLR2.

### EXAMPLE 5

To assess the effect of pimecrolimus on the innate immune response of the epidermis, cultures of normal human keratinocytes (NHEK) are stimulated with a concentration of 10 nM pimecrolimus, 1 uM tacrolimus, 1 ug/ml CsA, 25ng/ml rapamycin, or a combination of pimecrolimus and rapamycin, each of these with or without 0.1 μg/ml TLR2/6 ligand Malp-2. Cathelicidin mRNA expression is evaluated. Results are shown in the tables below.

TABLE 10
(means)

| | Experiment 1 | | | |
|---|---|---|---|---|
| | | with | Experiment 2 | |
| | without Malp-2 | Malp-2 | without Malp-2 | with Malp-2 |
| Control | 1.00 | 1.53 | 1.01 | 3.19 |
| Pimecrolimus | 1.33 | 2.98 | 2.40 | 3.07 |
| Tacrolimus | 0.67 | 5.47 | 3.34 | 0.77 |
| Cyclosporin A | 2.13 | 2.06 | 1.32 | 1.02 |
| Rapamycin | 5.13 | 4.03 | 5.85 | 12.38 |
| Rapamycin + | 0.68 | 1.69 | 9.07 | 26.48 |
| Pimecrolimus | | | | |

TABLE 11
(SD)

| | Experiment 1 | | | |
|---|---|---|---|---|
| | | with | Experiment 2 | |
| | without Malp-2 | Malp-2 | without Malp-2 | with Malp-2 |
| Control | 0.08 | 0.13 | 0.15 | 0.24 |
| Pimecrolimus | 0.15 | 1.07 | 0.30 | 0.22 |
| Tacrolimus | 0.04 | 0.83 | 0.42 | 0.28 |
| Cyclosporin A | 1.25 | 0.59 | 0.51 | 0.15 |
| Rapamycin | 0.68 | 0.44 | 1.13 | 1.82 |
| Rapamycin + | 0.29 | 0.24 | 4.03 | 6.94 |
| Pimecrolimus | | | | |

### EXAMPLE 6

To assess the effect of time dependent effects of pimecrolimus on the innate immune response of the epidermis, cultures of normal human keratinocytes (NHEK) are stimulated with 10 nM Pimecrolimus and low dose calciferol (1,25D3) 1 nM for different time points. mRNA expression of cathelicidin, CD14 and TLR2 is analyzed by qPCR. Results are shown in the tables below.

TABLE 12
(Cathelicidin)

| | | |
|---|---|---|
| 1.25D3 | PIM | PIM + 1.25D3 |

Means of N = 3

| | | | |
|---|---|---|---|
| 0 h | 1.00 | 1.20 | 1.04 |
| 8 h | 35.04 | 1.82 | 18.96 |
| 24 h | 134.03 | 1.75 | 196.50 |
| 48 h | 92.37 | 2.06 | 98.29 |

SD

| | | | |
|---|---|---|---|
| 0 h | 0.07 | 0.72 | 0.36 |
| 8 h | 3.25 | 0.42 | 4.80 |
| 24 h | 19.48 | 0.67 | 26.50 |
| 48 h | 10.12 | 1.28 | 10.68 |

TABLE 13
(CD14)

| | | |
|---|---|---|
| 1.25D3 | PIM | PIM + 1.25D3 |

Means of N = 3

| | | | |
|---|---|---|---|
| 0 h | 0.71 | 1.54 | 1.15 |
| 8 h | 38.35 | 1.46 | 6.81 |
| 24 h | 12.26 | 2.10 | 18.64 |
| 48 h | 0.93 | 1.04 | 1.64 |

SD

| | | | |
|---|---|---|---|
| 0 h | 0.71 | 1.54 | 1.15 |
| 8 h | 12.67 | 0.40 | 1.76 |
| 24 h | 1.57 | 0.94 | 4.16 |
| 48 h | 0.46 | 0.39 | 0.31 |

TABLE 14
(TLR2)

| | | |
|---|---|---|
| 1.25D3 | PIM | PIM + 1.25D3 |

Means of N = 3

| | | | |
|---|---|---|---|
| 0 h | 1.00 | 1.00 | 1.02 |
| 8 h | 1.32 | 0.64 | 0.37 |
| 24 h | 1.51 | 1.31 | 2.07 |
| 48 h | 1.55 | 1.51 | 1.10 |

SD

| | | | |
|---|---|---|---|
| 0 h | 0.07 | 0.06 | 0.26 |
| 8 h | 0.19 | 0.06 | 0.04 |
| 24 h | 0.18 | 0.12 | 0.50 |
| 48 h | 0.02 | 0.13 | 0.26 |

This demonstrates that the response of NHEK to Pimecrolimus and low dose calciferol (1,25D3 1 nM) exhibits an unexpected time dependent response. In particular, cathelicidin mRNA expression peaks after 24 hours after treatment with Pimecrolimus and 1,25D3, but is almost unaffected after 8 or 48 hours. Unexpectedly, 1,25D3-induced CD14 mRNA expression is suppressed by Pimecrolimus after 8 hours of treatment, but is enhanced by Pimecrolimus after 24 hours. Similar results seen for CD14 are also observed for TLR2 expression. These results show that pimecrolimus has a biphasic effect on innate immune genes dependent on time.

### EXAMPLE 7

To assess the effect of varying levels of pimecrolimus on the innate immune response of the epidermis, cultures of normal human keratinocytes (NHEK) are stimulated with 0 nM, 5 nM, 10 nM, 20 nM, 30 nM, 40 nM, 40 nM and 60 nM concentrations of pimecrolimus. Expression of cathelicidin mRNA expression is measured and evaluated accordingly to the procedures as detailed in Example 2 above. Results are detailed in the table below.

TABLE 15

| | | 5 nM | 10 nM | | | 60 |
|---|---|---|---|---|---|---|
| | Control | PIM | Pim | 20 nM Pim | 40 nM Pim | nM Pim |
| Mean | 1.01 | 1.46 | 2.26 | 82.72 | 3.37 | 1.78 |
| SD | 0.21 | 0.83 | 0.16 | 13.58 | 0.10 | 0.11 |

This demonstrates that the response of NHEK to Pimecrolimus exhibits an unexpected bell-shaped curve dosage response. In particular, cathelicidin MRNA expression peaks at a 20 nM concentration, with lower expression levels both below and above such concentration.

### EXAMPLE 8

To assess the effect of calciferol on varying levels of pimecrolimus on the innate immune response of the epidermis, cultures of normal human keratinocytes (NHEK) are stimulated with 0.01 nM, 0.1 nM, 1 nM and 10 nM concentrations of pimecrolimus with or without 1 nM concentrations of calciferol for 24 hours. Expression of cathelicidin mRNA and gene expression of TLR2 is measured and evaluated accordingly to the procedures as detailed in Example 2 above. Results are detailed in the table below.

TABLE 16
(cathelicidin)

| | no 1.25D3 | | | 1.25D3 | | |
|---|---|---|---|---|---|---|
| nM PIM | Mean | SD | N | Mean | SD | N |
| 0.01 | 1.04 | 1.13 | 3 | 4.56 | 0.33 | 3 |
| 0.1 | 0.47 | 0.44 | 3 | 4.19 | 1.14 | 3 |
| 1 | 1.61 | 0.76 | 3 | 10.92 | 8.76 | 3 |
| 10 | 9.81 | 3.02 | 3 | 99.45 | 39.49 | 3 |

TABLE 17
(TLR2)

| | no 1.25D3 | | | 1.25D3 | | |
|---|---|---|---|---|---|---|
| nM PIM | Mean | SD | N | Mean | SD | N |
| 0.01 | 0.73 | 0.81 | 3 | 0.82 | 0.21 | 3 |
| 0.1 | 0.59 | 0.58 | 3 | 0.57 | 0.50 | 3 |
| 1 | 0.98 | 0.09 | 3 | 1.07 | 0.26 | 3 |
| 10 | 1.80 | 0.02 | 2 | 3.94 | 1.21 | 2 |

This demonstrates at the 1 nM and 10 nM concentrations of pimecrolimus that the response of NHEK cathelicidin mRNA expression increases by greater than at least 2 times when exposed to pimecrolimus in conjunction with calciferol versus exposure to pimecrolimus alone.

1. A method for the treatment of diseases in a patient in which enhancement of the innate immune response can treat the disease, which method comprises administering to the patient a therapeutically effective amount of a composition comprising:
(a) at least one calcineurin inhibitor, mTOR inhibitor or non-immunosuppressive derivative, or a derivative, isomer, or pharmaceutically acceptable salt thereof; and
(b) optionally, at least one calciferol or LMW inhibitor, or a derivative, isomer, or pharmaceutically acceptable salt thereof.

2. The method of claim 1 wherein the calcineurin inhibitor is selected from the group consisting of tacrolimus, pimecrolimus, ascomycin, cyclosporin A, tacrolimus, cypermethrin, deltamethrin, fenvalerate, vocyclosporin and the derivatives, isomers and pharmaceutically acceptable salts thereof.

3. The method of claim 1 wherein the mTOR inhibitor is selected from the group consisting of rapamycin, rapamycin derivatives, everolimus, 7-tert-Butyl-6-(4-chloro-phenyl)-2-thioxo-2,3-dihydro-1H-pyrido[2,3-d]pyrimidin-4-one, SAR943, 40-O-alkyl-rapamycin derivatives, 32-deoxo-rapamycin derivatives, 32-hydroxy-rapamycin derivatives, 32-deoxorapamycin, 16-O-substituted rapamycin derivatives, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, rapamycin derivatives which are acylated at the oxygen group in position 40,40-[3-hydroxy-2-(hydroxy-methyl)-2-methylpropanoate]-rapamycin, rapamycin derivatives which are substituted in 40 position by heterocyclyl, 40-epi-(tetrazolyl)-rapamycin, rapalogs, 40-O-dimethylphosphinyl-rapamycin, AP23573, biolimus, 40-O-(2-ethoxy)ethyl-rapamycin, TAFA-93, AP23464, AP23675, AP23841, and the derivatives, isomers and pharmaceutically acceptable salts thereof.

4. The method of claim 1 wherein the immunosuppressive derivative is a compound of formula I to III 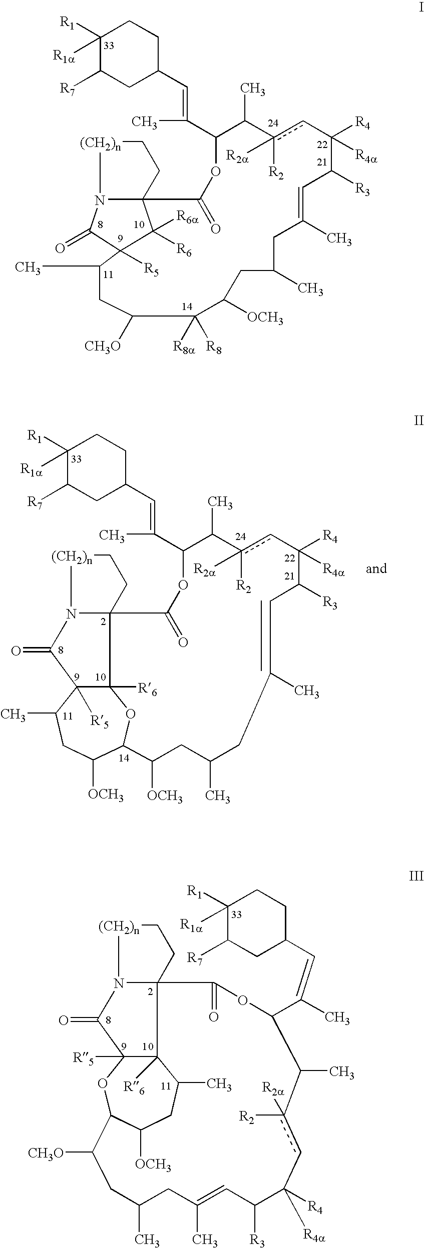
wherein the symbol [symbol] represents a single bond or, when R₂ₐ is absent, a double bond;
R₁ represents an optionally protected hydroxy group and R₁ₐ represents hydrogen;
or R₁ and R₁ₐ together represent oxo;
R₂ represents an optionally protected hydroxy group or together with R₄forms the —OC(═O)O— group, and R₂ₐ represents hydrogen or is absent;
whereby when the symbol [symbol] is a single bond,
R₂ together with R₂ₐ also represents oxo;
R₃ represents methyl, ethyl, n-propyl or allyl;
R₄ represents optionally protected hydroxy or together with R₂ forms the —OC(═O)O— group, and R₄ₐ represents hydrogen;
or R₄ together with R₄ₐ represents oxo;
R₅ represents alkoxycarbonyloxy, halogen, optionally protected hydroxy, lower alkoxy, acyloxy or a group —OC(═X)N(R₁₀)R₁₁;
or R₅together with R₆ₐ forms a group —OC(═X)N(R′₁₀)— attached with the nitrogen atom to the carbon atom carrying R₆ₐ, whereby X represents oxygen or sulfur, R₁₀ and R₁₁ independently represent hydrogen or lower alkyl or together with the nitrogen atom form a five- or six-membered ring optionally containing a second heteroatom such as nitrogen or oxygen, and R′₁₀ is hydrogen or lower alkyl;
or R₅ together with R₈ₐ represents oxy, whereby R₈ represents hydroxy;
R₆ represents hydroxy, and R₆ₐ represents hydrogen or together with R₅ forms a group —OC(═X)N(R′₁₀)— as defined above;
or R₆and R₆ₐ together represent oxo;
R′₅ represents optionally protected hydroxy, lower alkoxy or acyloxy and R′₆represents hydroxy;
or R′₅ and R′₆ together form the —OC(═O)O— group;
R″₅ represents hydroxy or lower alkoxy and R″.sub.6 represents hydroxy;
or R″₅and R″₆ together form the —OC(═O)O— group;
R₇represents methoxy or hydroxy;
R₈ represents an optionally protected hydroxy group, acyloxy, imidazolylcarbonyloxy or alkoxycarbonyloxy and R₈ₐ represents hydrogen;
or R₈ represents hydroxy and R₈ₐ together with R₅ represents oxy;
or R₈ together with R₈ₐ represents oxo; and
n represents 1 or 2;
and the derivatives, isomers and pharmaceutically acceptable salts thereof.

5. The method of claim 1 wherein the calciferol is selected from the group consisting of calciferol, calcipotriol, calcitriol, cholecalciferol, 22,23-dihydroergocalciferol, 25-hydroxycholecalciferol, 25-hydroxyergocalciferol, maxacalcitol, falecalcitol, falecalcitriol and tacalcitol, and the derivatives, isomers and pharmaceutically acceptable salts thereof.

6. The method of claim 1 wherein the LMW inhibitor is a compound of the formula 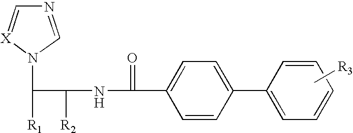
wherein either R₁ is phenyl, naphthyl, thienyl or pyridyl, or phenyl, naphthyl, thienyl or pyridyl monosubstituted by halogen, C₁-C₄ alkoxy, C₁-C₄ alkyl, di-C₁-C₄ alkylamino or cyano and
R₂ is hydrogen; or
R₁ is hydrogen and
R₂ is pyridyl or 2-(5-chloro)pyridyl;
R₃ is hydrogen, halogen, C₁-C₄ alkyl, C₁-C₄ alkoxy, cyano, C₁-C₄ alkoxycarbonyl, C₁-C₄ alkylcarbonyl, amino or di-C₁-C₄ alkylamino; and
X is CH;
and the derivatives, isomers and pharmaceutically acceptable salts thereof.

7. The method of claim 1 wherein the disease is selected from the group consisting of acne, atopic dermatitis, wound infections, MRSA, viral skin infections, HSV, HPV, Poxvirus, onychomycosis, viral and bacterial infections of the oro-pharynx and respiratory tract, RSV, mycoplasm, infections of urinary tract, Chlamydia, viral and bacterial infections of the GI tract, psoriasis, contact dermatitis, eczematous dermatitises, seborrhoeic dermatitis, Lichen planus, Pemphigus, bullous Pemphigoid, Epidermolysis bullosa, urticaria, angioedemas, vasculitides, erythemas, cutaneous eosinophilias, Lupus erythematous, Alopecia areata, cornefied epithelia, gingivitis, oro-pharyngitis, inflammatory bowel disease, Crohns disease, recurrent vaginitis (E. coli), urethritis)), and conjunctiva.

8. A pharmaceutical composition for enhancing an innate immune response in an patient comprising a therapeutically effective amount of:
(a) at least one calcineurin inhibitor, mTOR inhibitor or non-immunosuppressive derivative, or a derivative, isomer, or pharmaceutically acceptable salt thereof; and
(b) optionally, at least one calciferol or LMW inhibitor, or a derivative, isomer, or pharmaceutically acceptable salt thereof.

9. The composition of claim 8 wherein the calcineurin inhibitor is selected from the group consisting of tacrolimus, pimecrolimus, ascomycin, cyclosporin A, tacrolimus, cypermethrin, deltamethrin, fenvalerate, vocyclosporin and the derivatives, isomers and pharmaceutically acceptable salts thereof.

10. The composition of claim 8 wherein the mTOR inhibitor is selected from the group consisting of rapamycin, rapamycin derivatives, everolimus, 7-tert-Butyl-6-(4-chloro-phenyl)-2-thioxo-2,3-dihydro-1H-pyrido[2,3-d]pyrimidin-4-one, SAR943, 40-O-alkyl-rapamycin derivatives, 32-deoxo-rapamycin derivatives, 32-hydroxy-rapamycin derivatives, 32-deoxorapamycin, 16-O-substituted rapamycin derivatives, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, rapamycin derivatives which are acylated at the oxygen group in position 40,40-[3-hydroxy-2-(hydroxy-methyl)-2-methylpropanoate]-rapamycin, rapamycin derivatives which are substituted in 40 position by heterocyclyl, 40-epi-(tetrazolyl)-rapamycin, rapalogs, 40-O-dimethylphosphinyl-rapamycin, AP23573, biolimus, 40-O-(2-ethoxy)ethyl-rapamycin, TAFA-93, AP23464, AP23675, AP23841, and the derivatives, isomers and pharmaceutically acceptable salts thereof.

11. The composition of claim 8 wherein the immunosuppressive derivative is a compound of formula I to III 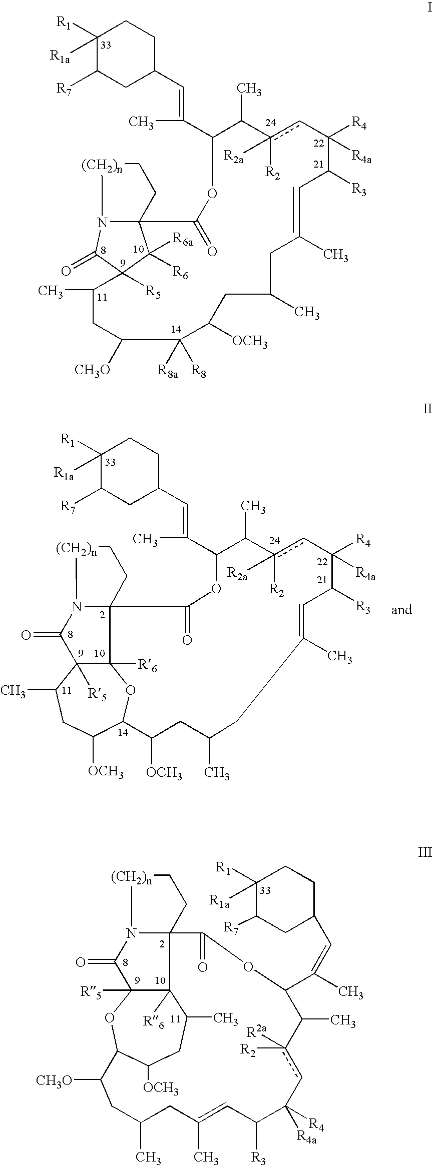
wherein the symbol [symbol] represents a single bond or, when R₂ₐ is absent, a double bond;
R₁ represents an optionally protected hydroxy group and R₁ₐ represents hydrogen;
or R₁ and R₁ₐ together represent oxo;
R₂ represents an optionally protected hydroxy group or together with R₄ forms the —OC(═O)O— group, and R₂ₐ represents hydrogen or is absent;
whereby when the symbol [symbol] is a single bond,
R₂ together with R₂ₐ also represents oxo;
R₃ represents methyl, ethyl, n-propyl or allyl;
R₄ represents optionally protected hydroxy or together with R₂ forms the —OC(═O)O— group, and R₄ₐ represents hydrogen;
or R₄ together with R₄ₐ represents oxo;
R₅ represents alkoxycarbonyloxy, halogen, optionally protected hydroxy, lower alkoxy, acyloxy or a group —OC(═X)N(R₁₀)R₁₁;
or R₅ together with R₆ₐ forms a group —OC(═X)N(R′₁₀)— attached with the nitrogen atom to the carbon atom carrying R₆ₐ, whereby X represents oxygen or sulfur, R₁₀ and R₁₁ independently represent hydrogen or lower alkyl or together with the nitrogen atom form a five- or six-membered ring optionally containing a second heteroatom such as nitrogen or oxygen, and R′₁₀ is hydrogen or lower alkyl;
or R₅ together with R₈ₐ represents oxy, whereby R₈ represents hydroxy;
R₆ represents hydroxy, and R₆ₐ represents hydrogen or together with R₅ forms a group OC(═X)N(R′₁₀)— as defined above;
or R₆ and R₆ₐ together represent oxo;
R′₅ represents optionally protected hydroxy, lower alkoxy or acyloxy and R′₆represents hydroxy;
or R′₅ and R′₆ together form the —OC(═O)O— group;
R″₅ represents hydroxy or lower alkoxy and R″.sub.6 represents hydroxy;
or R″₅and R″₆ together form the —OC(═O)O— group;
R₇represents methoxy or hydroxy;
R₈ represents an optionally protected hydroxy group, acyloxy, imidazolylcarbonyloxy or alkoxycarbonyloxy and R₈ₐ represents hydrogen;
or R₈ represents hydroxy and R₈ₐ together with R₅ represents oxy;
or R₈ together with R₈ₐ represents oxo; and
n represents 1 or 2;
and the derivatives, isomers and pharmaceutically acceptable salts thereof.

12. The composition of claim 8 wherein the calciferin is selected from the group consisting of calciferol, calcipotriol, calcitriol, cholecalciferol, 22,23-dihydroergocalciferol, 25-hydroxycholecalciferol, 25-hydroxyergocalciferol, maxacalcitol, falecalcitol, falecalcitriol and tacalcitol, and the derivatives, isomers and pharmaceutically acceptable salts thereof.

13. The composition of claim 8 wherein the LMW inhibitor is a compound of the formula 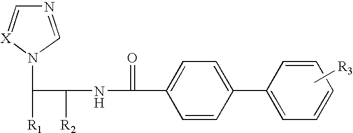
wherein either R₁ is phenyl, naphthyl, thienyl or pyridyl, or phenyl, naphthyl, thienyl or pyridyl monosubstituted by halogen, C₁-C₄ alkoxy, C₁-C₄ alkyl, di-C₁-C₄ alkylamino or cyano and
R₂ is hydrogen; or
R₁ is hydrogen and
R₂ is pyridyl or 2-(5-chloro)pyridyl;
R₃ is hydrogen, halogen, C₁-C₄ alkyl, C₁-C₄ alkoxy, cyano, C₁-C₄ alkoxycarbonyl, C₁-C₄ alkylcarbonyl, amino or di-C₁-C₄ alkylamino; and
X is CH;
and the derivatives, isomers and pharmaceutically acceptable salts thereof.

14. A method for enhancing an innate immune response in an patient comprising administering to an patient a therapeutically effective amount of a composition comprising
(a) at least one calcineurin inhibitor, mTOR inhibitor or non-immunosuppressive derivative, or a derivative, isomer, or pharmaceutically acceptable salt thereof; and
(b) optionally, at least one calciferol or LMW inhibitor, or a derivative, isomer, or pharmaceutically acceptable salt thereof.

15. The method of claim 14 wherein the calcineurin inhibitor is selected from the group consisting of tacrolimus, pimecrolimus, ascomycin, cyclosporin A, tacrolimus, cypermethrin, deltamethrin, fenvalerate, vocyclosporin and the derivatives, isomers and pharmaceutically acceptable salts thereof.

16. The method of claim 14 wherein the mTOR inhibitor is selected from the group consisting of rapamycin, rapamycin derivatives, everolimus, 7-tert-Butyl-6-(4-chloro-phenyl)-2-thioxo-2,3-dihydro-1H-pyrido[2,3-d]pyrimidin-4-one, SAR943, 40-O-alkyl-rapamycin derivatives, 32-deoxo-rapamycin derivatives, 32-hydroxy-rapamycin derivatives, 32-deoxorapamycin, 16-O-substituted rapamycin derivatives, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, rapamycin derivatives which are acylated at the oxygen group in position 40,40-[3-hydroxy-2-(hydroxy-methyl)-2-methylpropanoate]-rapamycin, rapamycin derivatives which are substituted in 40 position by heterocyclyl, 40-epi-(tetrazolyl)-rapamycin, rapalogs, 40-O-dimethylphosphinyl-rapamycin, AP23573, biolimus, 40-O-(2-ethoxy)ethyl-rapamycin, TAFA-93, AP23464, AP23675, AP23841, and the derivatives, isomers and pharmaceutically acceptable salts thereof.

17. The method of claim 14 wherein the immunosuppressive derivative is a compound of formula I to III 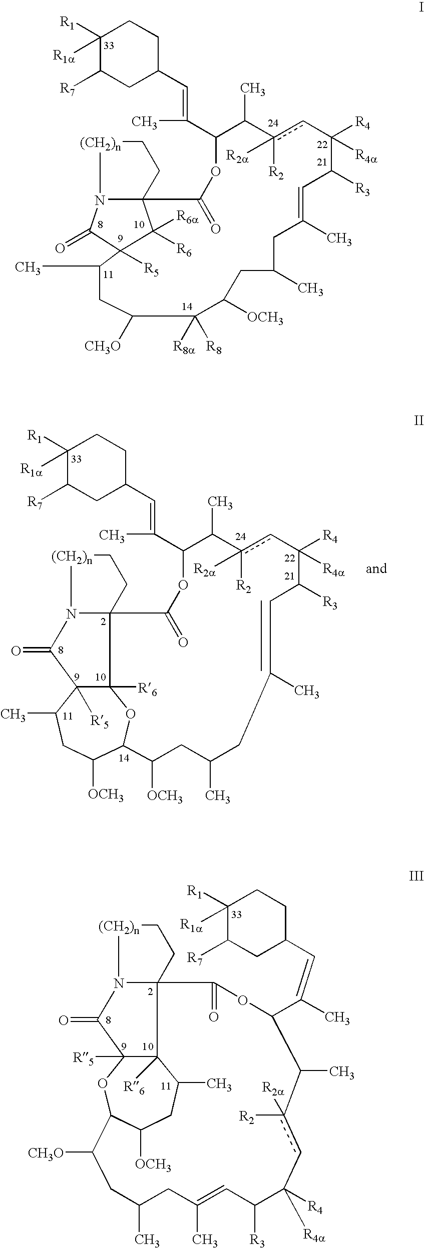
wherein the symbol [symbol] represents a single bond or, when R₂ₐ is absent, a double bond;
R₁ represents an optionally protected hydroxy group and R₁ₐ represents hydrogen;
or R₁ and R₁ₐ together represent oxo;
R₂ represents an optionally protected hydroxy group or together with R₄forms the —OC(═O)O— group, and R₂ₐ represents hydrogen or is absent;
whereby when the symbol [symbol] is a single bond,
R₂ together with R₂ₐ also represents oxo;
R₃ represents methyl, ethyl, n-propyl or allyl;
R₄ represents optionally protected hydroxy or together with R₂ forms the —OC(═O)O— group, and R₄ₐ represents hydrogen;
or R₄ together with R₄ₐ represents oxo;
R₅ represents alkoxycarbonyloxy, halogen, optionally protected hydroxy, lower alkoxy, acyloxy or a group —OC(═X)N(R₁₀)R₁₁;
or R₅ together with R₆ₐ forms a group —OC(═X)N(R′₁₀)— attached with the nitrogen atom to the carbon atom carrying R₆ₐ, whereby X represents oxygen or sulfur, R₁₀ and R₁₁ independently represent hydrogen or lower alkyl or together with the nitrogen atom form a five- or six-membered ring optionally containing a second heteroatom such as nitrogen or oxygen, and R′₁₀ is hydrogen or lower alkyl;
or R₅ together with R₈ₐ represents oxy, whereby R₈ represents hydroxy;
R₆ represents hydroxy, and R₆ₐ represents hydrogen or together with R₅ forms a group —OC(═X)N(R′₁₀)— as defined above;
or R₆and R₆ₐ together represent oxo;
R′₅ represents optionally protected hydroxy, lower alkoxy or acyloxy and R′₆represents hydroxy;
or R′₅ and R′₆ together form the —OC(═O)O— group;
R″₅ represents hydroxy or lower alkoxy and R″.sub.6 represents hydroxy;
or R″₅and R″₆ together form the —OC(═O)O— group;
R₇represents methoxy or hydroxy;
R₈ represents an optionally protected hydroxy group, acyloxy, imidazolylcarbonyloxy or alkoxycarbonyloxy and R₈ₐ represents hydrogen;
or R₈ represents hydroxy and R₈ₐ together with R₅ represents oxy;
or R₈ together with R₈ₐ represents oxo; and
n represents 1 or 2;
and the derivatives, isomers and pharmaceutically acceptable salts thereof.

18. The method of claim 14 wherein the calciferol is selected from the group consisting of calciferol, calcipotriol, calcitriol, cholecalciferol, 22,23-dihydroergocalciferol, 25-hydroxycholecalciferol, 25-hydroxyergocalciferol, maxacalcitol, falecalcitol, falecalcitriol and tacalcitol, and the derivatives, isomers and pharmaceutically acceptable salts thereof.

19. The method of claim 14 wherein the LMW inhibitor is a compound of the formula 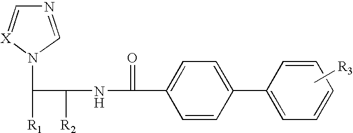
wherein either R₁ is phenyl, naphthyl, thienyl or pyridyl, or phenyl, naphthyl, thienyl or pyridyl monosubstituted by halogen, C₁-C₄ alkoxy, C₁-C₄ alkyl, di-C₁-C₄ alkylamino or cyano and
R₂ is hydrogen; or
R₁ is hydrogen and
R₂ is pyridyl or 2-(5-chloro)pyridyl;
R₃ is hydrogen, halogen, C₁-C₄ alkyl, C₁-C₄ alkoxy, cyano, C₁-C₄ alkoxycarbonyl, C₁-C₄ alkylcarbonyl, amino or di-C₁-C₄ alkylamino; and
X is CH;
and the derivatives, isomers and pharmaceutically acceptable salts thereof.

20. The method of claim 14 wherein the disease is selected from the group consisting of acne, atopic dermatitis, wound infections, MRSA, viral skin infections, HSV, HPV, Poxvirus, onychomycosis, viral and bacterial infections of the oro-pharynx and respiratory tract, RSV, mycoplasm, infections of urinary tract, Chlamydia, viral and bacterial infections of the GI tract, psoriasis, contact dermatitis, eczematous dermatitises, seborrhoeic dermatitis, Lichen planus, Pemphigus, bullous Pemphigoid, Epidermolysis bullosa, urticaria, angioedemas, vasculitides, erythemas, cutaneous eosinophilias, Lupus erythematous, Alopecia areata, cornefied epithelia, gingivitis, oro-pharyngitis, inflammatory bowel disease, Crohns disease, recurrent vaginitis (E. coli), urethritis)), and conjunctiva.

21. (canceled)
